SEQUENCE LISTING (6 sequences)

SEQ ID NO: 1 - DNA, 23 bp, synthetic construct
  source 1..23: mol_type = other DNA
gccgtagggtcgaaatggtcaag

SEQ ID NO: 2 - DNA, 22 bp, synthetic construct
  source 1..22: mol_type = other DNA
ttgctacaccctcggatccgaa

SEQ ID NO: 3 - DNA, 20 bp, synthetic construct
  source 1..20: mol_type = other DNA
aagagtgcaggcaaaacgat

SEQ ID NO: 4 - DNA, 21 bp, synthetic construct
  source 1..21: mol_type = other DNA
caacaatgcttttctggcttc

SEQ ID NO: 5 - DNA, 21 bp, synthetic construct
  source 1..21: mol_type = other DNA
gttaacactacgacctccacg

SEQ ID NO: 6 - DNA, 20 bp, synthetic construct
  source 1..20: mol_type = other DNA
acctcgagacttacgacccg

(19) United States
(12) Patent Application Publication
(10) Pub. No.: US 2026/0083160 A1
(43) Pub. Date: Mar. 26, 2026

(54) EDIBLE AERIAL MYCELIUM, METHODS FOR THEIR PREPARATION AND FOOD PRODUCTS COMPRISING THE SAME

(71) Applicants: MUSHFOODS LTD, Ashdod (IL); MIGAL GALILEE RESEARCH INSTITUTE LTD, Kiryat Shmona (IL)

(72) Inventors: Shalom DANIEL, Ashdod (IL); Jonathan CASPI, Ashdod (IL); Avinoam SABATO, Ashdod (IL); Idan PEREMAN, Kiryat Shmona (IL); Dan LEVANON, Kiryat Shmona (IL); Nirit EZOV, Kiryat Shmona (IL)

(21) Appl. No.: 19/125,893

(22) PCT Filed: Nov. 5, 2023
(86) PCT No.: PCT/IL2023/051135

§ 371 Date: Apr. 30, 2025

Related U.S. Application Data
(60) Provisional application No. 63/423,200, filed on Nov. 7, 2022.

(51) Int. Cl. A23L 31/00; A23J 3/20; A23J 3/22; A23L 13/40; A23L 27/00; A23L 33/21; C12N 1/14

(52) U.S. Cl. CPC A23L 31/00; A23J 3/20; A23J 3/22; A23L 13/428; A23L 13/43; A23L 13/46; A23L 27/88; A23L 33/21; C12N 1/14

(57) ABSTRACT

Provided herein are edible aerial mycelium, compositions including the same and methods for their preparation. Further provided are various food products including the aerial mycelium, such as hybrid food products containing mixtures of aerial mycelium and meat of various origin.

### FIELD OF THE INVENTION

The present invention, in some embodiments thereof, relates to edible aerial mycelium, compositions including the same and methods for their preparation. Further provided are various food products including the aerial mycelium.

### BACKGROUND OF THE INVENTION

Mycelium is a root-like system of the fungus kingdom. It is consistent of fungal cells, very similar to those of the above-the-ground fruit bodies (a.k.a. “mushrooms”). In nature, the mycelial parts hold more than 90% of the species total biomass. It grows rapidly on rotten trees and branches as well as on a variety of organic waste. Fungi, in general, are highly nutritious, contain a full profile of essential amino acids and are rich in fiber, vitamins and minerals.

Fungi mycelium is a target product for many companies. In its simplest form, it is grown as a fermented tissue on a variety of edible substrates and is packed, together with the substrate, as a whole product.

Separating the mycelial tissue from its substrate is more challenging and requires the development of specific methodology. The most efficient method today is liquid fermentation. Fungi cells are mixed in large fermenters of sugar and amino acid rich media, under controlled conditions to produce large biomass which is then harvested as a 100% fungi biomass. These systems are relatively easy to scale up and are the most cost effective in terms of biomass per $. However, liquid fermentation is not suitable for edible mushroom mycelium and thus are used to grow yeast and molds. The end result is compromised in terms of flavor and aromas and faces a number of regulatory issues.

Solid phase fermentation is preferred for edible fungi mycelium. It utilizes cost effective plant harvest waste or even food-grade waste from a large variety of industries. The challenge, however, is separating the mycelial tissue from the substrate.

Exemplary publications related to growth and preparation of mycelium include, US2012315687, KR1020200091788, US20090098620, Avni, et al., “Olive Mill Waste Enhances-Glucan Content in the Edible Mushroom Pleurotus eryngii”, Int. J. Mol. Sci. 2017, 18, 1564; doi: 10.3390/ijms18071564, CN106434373, JP1998287584, KR1020050005133, KR1020170069547, US20190249211, KR1020200108748, WO2021123831, WO202061502, WO2020092306, Drori, et al., “Extracts from Lentimula edodes (Shiitake) Edible Mushrooms Enriched with Vitamin D Exert an Anti-Inflammatory Hepatoprotective Effect”, J Med Food 19 (4) 2016, 383-389.

Therefore, there is a need in the art for edible aerial mycelium having a controlled nutritional profile and processes for growing such nutritionally enhanced aerial mycelium.

### SUMMARY OF THE INVENTION

According to some embodiments, provided herein are improved edible aerial mycelium having an enhanced nutritional profile, methods for their preparation and composition including the same.

In some embodiments, there is provided a solid-state fermentation process to facilitate the growth of essentially clean mycelium, which allows controlling the nutritional profile of the mycelium.

According to some embodiments, the advantageous mycelium disclosed herein and composition comprising the same can be used in various products, in particular, for food products.

According to some embodiments, there are provided food products which include the mycelium, said products exhibit enriched nutritional values as compared to products devoid of the mycelium.

According to some embodiments, the advantageous mycelium disclosed herein, can be used in various food products compositions, including, for example, meat substitutes food products, mixture with other plant-based meat substitutes and/or mixture with various meat products, to thereby improve taste, appearance and/or nutritional values.

According to some embodiments, there is provided herein a mycelium derived from a cultivation substrate comprising olive mill solid waste (OMSW) at a concentration of about 30-60%, said mycelium comprises at least about 35% w/w of total Glucan and at least about 6.5% w/w of total protein. Optionally, the mycelium may further comprise at least about 2.8% w/w Nitrogen. Optionally, the mycelium may further comprise at least about 40 μg/g of Vitamin D. Optionally, the Glucans may comprise at least about 19% beta-Glucan and at least about 5% alpha-Glucan.

According to some embodiments, the mycelium may be aerial mycelium. Optionally, the yield of the mycelium may be at least about 25% w/w. Optionally, the yield of the mycelium may be the result of multiple growth cycles.

According to some embodiments, the mycelium may be from the species Pleurotus osteratus, Pleurotus eryngii, Pleurotus sp. Pleurotus.colombinus, Pleurotus pulmonarius, Pleurotus sajor caju, Pleurotus cornucopiae, or Pleurotus salmoneo.

According to some embodiments, the mycelium may be for use as a binder of a food product mixture. Optionally, the mycelium may be in powdered form. Optionally, the mycelium may be milled to a particle size of about 0.2 mm to 200 mm.

According to some embodiments, a composition may comprise a mixture of at least one aerial mycelium. Optionally, a composition may comprise at least two aerial mycelium, optionally each derived from a different species. Optionally, at least one of the species may be selected from Pleurotus osteratus, Pleurotus eryngii, Pleurotus sp. Pleurotus.colombinus, Pleurotus pulmonarius, Pleurotus sajor caju, Pleurotus cornucopiae, or Pleurotus salmoneo.

According to some embodiments, a food product comprising a mixture of mycelium and at least partially ground meat. Optionally, fruit bodies may be added to the food product. Optionally, the mycelium may be grinded prior to mixing with the meat. Optionally, the aerial mycelium may be mixed with water at a ratio of about 10:90, about 20:80, about 25:75, about 40:60, about 50:50, about 60:40, about 75:25 about 80:20, or about 90:10 prior to mixing with the meat. Optionally, the meat may be selected from poultry, beef, pork, lamb, fish, seafood, cultured meat (cell culture), etc. Optionally, further ingredients may be added, such as one or more of: oil, flavor enhancer and colorant.

According to some embodiments, the weight ratio between the mycelium and the meat may be in the range, about 10:90, about 20:80, about 25:75, about 40:60, about 50:50, about 60:40, about 75:25 about 80:20, or about 90:10 w/w.

According to some embodiments, the food product may comprise at least about 2% dietary fibers (w/w).

According to some embodiments, the food product may comprise less than about 15% fat (w/w).

According to some embodiments, the food product may comprise less than about 5% saturated fat (w/w).

According to some embodiments, the food product may have a water content of about 65-85% w/w.

According to some embodiments, the food product may be stable and/or moldable. Optionally, the food product may be essentially homogenous.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1: Exemplary image of fungal fruiting bodies with mycelium within substrate, in accordance with some embodiments;

FIG. 2A: Exemplary image of mycelium sheets grown on substrate in a growth chamber, in accordance with some embodiments;

FIG. 2B: Exemplary image of harvested mycelium, in accordance with some embodiments;

FIG. 2C: Exemplary image of cross-section of harvested mycelium, in accordance with some embodiments;

FIG. 3: Examples of mycelium (“mush”) powder or chips with and without fruit bodies and exemplary images thereof, for use in various food products, in accordance with some embodiments;

FIG. 4: Flow diagram of method for growing fungal matter, in accordance with some embodiments;

FIG. 5: Flow diagram of a method for growing fruiting bodies, in accordance with some embodiments; and

FIG. 6: Flow diagram of a method for growing mycelial matter, in accordance with some embodiments.

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

An aspect of some embodiments of the current invention relates to growing mycelial tissue which may be easily separated from its substrate.

In an embodiment, a variety of fungal species may be used to provide mycelium. Preferably, a variety of Pleurotus species may be used to provide the mycelium. Optionally, the fungal species may be selected from an edible species of the genus Agrocybe, Albatrellus, Amillaria, Agaricus, Bondarzewia, Cantharellus, Cerioporus, Climacodon, Cordyceps, Fistulina, Flammulina, Fomes, Fomitopsis, Fusarium, Grifola, Herecium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces or Wolfiporia.

In an embodiment, the mycelial tissue may be grown under conditions which maintain a high level of humidity (e.g., above 90%), high level of CO₂ (e.g., approximately 5%) and minimum light to mimic underground conditions. Optionally, the growth conditions may be selected to encourage the fungi to develop more mycelial tissue and/or prevent the development of fruit bodies (e.g., mushrooms, toadstools, etc.). Optionally, such conditions may result in a semi-unified layer of mycelium. Optionally, the semi-unified layer of mycelium may cover the surface of the substrate. Optionally, the semi-unified layer of mycelium may be raised to a sheet of a few centimeters. Optionally, the sheet of 100% fungi material may be easily separated from the surface of the substrate and can be furthered processed. Optionally, the mycelium produced may be aerial mycelium.

In an embodiment, a sealed solid-state fermentation chamber may be used to grow clean mycelium. Optionally, the humidity, gas mixture, temperature, light and/or any combination thereof may be controlled in the sealed fermentation chamber, for example approximately 5% CO₂, minimal light, above 95% humidity, etc.

In an embodiment, the mycelium sheets may be grown in trays. Optionally, the trays may be placed in sealed fermentation chambers and/or growth rooms. Optionally, the humidity, gas mixture, temperature, light and/or any combination thereof may be controlled in the sealed fermentation chamber and/or growth room. Optionally, scale up of the process may involve the addition of more trays and/or larger fermentation chambers and/or larger growth rooms.

In an embodiment, the substate may comprise plant and food industry wastes. Optionally, the substrate may comprise olive mill solid waste (OMSW), sawdust (e.g., eucalyptus sawdust, soft wood sawdust, ground tree branches (interchangeable with sawdust), etc.), beer industry waste (e.g., malt waste, beer cereal waste), coffee waste (e.g., from coffee capsules, coffee factories, etc.), soy waste from tofu industry, wheat bran, corn stover, etc. and/or any combination thereof.

In some embodiments, the substrate may include between about 10-95%, and/or between about 20-90%, and/or between about 30-80%, and/or between about 40-70%, and/or between about 30-60%, and/or between about 50-60% of OMSW. In some embodiments, the amount of OMSW may be between about 30-60%. In some embodiments, the amount of OMSW may be between about 25-45%. In some embodiments, the amount of OMSW may be between about 30-40%. Each possibility is a separate embodiment.

In some embodiments, the substrate may include between about 10-95%, and/or between about 20-90%, and/or between about 30-80%, and/or between about 40-70%, and/or between about 50-60% of sawdust/ground branches (such as, eucalyptus sawdust/ground branches). In some embodiments, the amount of sawdust may be between about 25-45%. In some embodiments, the amount of sawdust may be between about 30-40%. Each possibility is a separate embodiment.

In some embodiments, the substrate may include between about 10-95%, and/or between about 20-90%, and/or between about 30-80%, and/or between about 40-70%, and/or between about 50-60% of cereal, such as, beer cereal. In some embodiments, the amount of cereal may be between about 20-50%. In some embodiments, the amount of cereal may be between about 30-45%. Each possibility is a separate embodiment.

In some exemplary embodiments, the substrate may include about 30-45% OMSW, about 25-45% sawdust (such as, Eucalyptus sawdust), and about 30-45% cereal (Such as, beer cereal). In some embodiments, the substrate may further include ground coffee.

In some exemplary embodiments, the substrate may include about 25-45% OMSW, about 15-45% sawdust (such as, Eucalyptus sawdust), and about 30-50% cereal (Such as, beer cereal). In some embodiments, the substrate may further include ground coffee.

In an embodiment, the use of OMSW in the substrate may increase the amount of glucan in the mycelium as compared to the absence of OMSW in the substrate. Optionally, the ratio of the OMSW to other organic waste may affect ratio of alpha to beta glucans. Optionally, the mycelium may comprise at least about 5%, at least about 10%, at least about 15%, at least about 20%, at least about 25%, at least about 30%, at least about 35%, at least about 40%, at least about 50% dry weight (dw)/dw of total glucan. Preferably, the mycelium may comprise at least about 35% dw/dw of total glucan.

In an embodiment, alpha and beta glucan synthase gene expression may positively correlate with amount of OMSW in the substrate. Optionally, the substrate may comprise between about 10-95%, and/or between about 20-80%, and/or between about 30-60%, and/or between about 50-60% of OMSW for glucan synthase gene expression. Optionally, about 30-60% of OMSW in the substrate may be optimal for glucan synthase gene expression. Optionally, the glucans level may be a function of substrate content. Optionally, the optimal component ratio of the substrate to maximize glucans content may be different than optimal ratio in the substrate to maximize mycelium growth. Optionally, the glucans in the mycelium may comprises at least about 10%, at least about 15%, at least about 20%, at least about 25% beta-glucan and/or at least about 4%, at least about 5%, at least about 5.5%, at least about 6%, at least about 6.5% alpha-glucan. Preferably, at least about 19% beta-glucan and/or at least about 5% alpha-glucan.

In an embodiment, the mycelium may comprise at least about 4%, at least about 5%, at least 5.5%, at least about 6%, at least about 6.5%, at least about 7%, at least about 7.5%, at least about 8% w/w of total protein. Preferably, the mycelium may comprise at least about 6.5% w/w of total protein.

In an embodiment, the mycelium may comprise at least about 1.5%, at least about 2%, at least about 2.5%, at least about 3%, at least about 3.5%, at least about 4%, at least about 4.5%, at least about 5%, at least about 7% w/w of albumin and/or globulin.

In an embodiment, the mycelium may comprise at least about 1%, at least about 1.5%, at least about 2%, at least about 2.5%, at least about 3%, at least about 3.5, at least about 4%, at least about 4.5%, at least about 5%, at least about 6%, at least about 7%, at least about 10%, at least about 15% dw/dw of nitrogen. Preferably, the mycelium may comprise at least about 2.8% dw/dw of nitrogen. Optionally, the amount of nitrogen in the mycelium may be an indication of the amount of protein present in the mycelium.

In an embodiment, cost of biomass growth may be reduced by multiple growth rounds. Optionally, multiple growth rounds may increase efficiency and/or yields and/or dramatically decrease costs. Optionally, the same inoculate and/or substrate may be used in multiple growth rounds. Optionally, the additives may be added to the substrate to maintain biomass yield. Optionally, the additives may comprise nitrogen-based fertilizers.

In an embodiment, the total yield of mycelium after all growth cycles may be at least about 10%, at least about 15%, at least about 20%, at least about 25%, at least about 30%, at least about 35%, at least about 40%, at least about 50%, at least about 60%, at least about 70%, at least about 80% w/w. Preferably, the total yield of mycelium after all growth cycles may be at least about 25% w/w.

In an embodiment, biomass mycelium may be used as a meat substitute and/or supplement and/or replacement and/or extender. Optionally, biomass mycelium may be used alone as a meat substitute. Optionally, biomass mycelium may be mixed with an animal meat (from livestock, or cultured meat (i.e., cultured animal cells)) to extend and/or supplement the animal product. Optionally, biomass mycelium may be mixed with other plant-based meat substitutes. Optionally, the mycelium may be mixed with other fungal products (e.g., fruit bodies, etc.). Optionally, the color and/or flavor and/or texture and/or stickiness of the mycelium may be adjusted for use in food industry.

In an embodiment, the mycelium biomass may be ground for use in the food industry, e.g., ground to a fine powder, milled to the same texture as ground and/or partially ground meat. Optionally, the meat may be selected from: poultry, beef, pork, lamb, fish, seafood, cultured meat (cell culture or tissue culture), etc. Optionally, the plant-based meat substitute may be legume based, soy based, wheat based, cruciferous vegetable based, grain based, etc. and/or any combination thereof. Optionally, the mycelium may be ground to a particle size of between about 0.2 mm to 200 mm, between about 0.5 mm to 150 mm, between about 0.75 mm to 100 mm, between about 1 mm to 75 mm, or between about 1.5 mm to 50 mm, as determined by sieving with a set of sieves. Optionally, the mycelium may be ground to powder (e.g., with a particle size between about 0.2 mm to 200 mm, between about 0.5 mm to 150 mm, between about 0.75 mm to 100 mm, between about 1 mm to 75 mm) prior to addition to the meat and/or a plant-based meat substitute. Optionally, the food product comprising the mycelium and the meat and/or plant-based meat substitute may be a an essentially homogenous mixture.

In some embodiments, the mixture may comprise between about 10-95%, and/or between about 20-80%, and/or between about 30-70%, and/or between about 40-60%, and/or between about 50-60% of ground mycelium. Optionally, the weight ratio between the mycelium and the meat may be in the range, about 10:90, about 20:80, about 25:75, about 40:60, about 50:50, about 60:40, about 75:25 about 80:20, or about 90:10 w/w.

In an embodiment, the mycelium may be mixed with water prior to mixing with the meat and/or plant-based meat substitute. Optionally, the weight ratio between the mycelium and the meat may be in the range, about 10:90, about 20:80, about 25:75, about 40:60, about 50:50, about 60:40, about 75:25 about 80:20, or about 90:10 w/w.

In an embodiment, the mycelium may be marinated, mixed with various additional fungi, milled together with meat, marinated, texturized, wet, etc. to improve the flavor, texture, palatability, color, etc. Optionally, the mycelium mat be mixed with an oil, flavor enhancer and/or colorant.

In an embodiment, the ground mycelium may be mixed with poultry, beef, pork, lamb, fish, seafood, cultured meat (i.e., cell culture) and/or any combination thereof. Optionally, mixing the mycelium with a meat and/or plant-based meat substitute may improve the nutritional value of the food, e.g., increasing the amount of dietary fiber, carbohydrates, minerals, vitamins, glucan, etc. and reducing the amount of fats, in particular saturated fats, etc. optionally, the mixture may comprise at least two aerial mycelium, optionally each derived from a different species. Optionally, the species may be Pleurotus osteratus, Pleurotus eryngii, Pleurotus sp. Pleurotus.colombinus, Pleurotus pulmonarius, Pleurotus sajor caju, Pleurotus cornucopiae, or Pleurotus salmoneo. Optionally, at least one of the species may be Pleurotus osteratus.

In an embodiment, the vitamin and/or mineral content and/or protein and/or fiber content of mycelium may be altered by altering the substrate composition, e.g., by addition of additives to the substrate, exposure to UV light, etc. Optionally, adjusting the composition of the growth substrate (e.g., before and/or during growth) and/or UV treatment (e.g., at the end of growth) controlled production of a raw material with health functional properties may be produced. Optionally, the mycelium may comprise at least about 10 μg/g, about 20 μg/g, about 30 μg/g, about 40 μg/g, about 50 μg/g, about 60 μg/g, about 70 μg/g, about 80 μg/g, about 90 μg/g, about 100 μg/g, about 150 μg/g, about 200 μg/g of Vitamin D. Optionally, the mycelium may preferably comprise between 40-60 μg/g of Vitamin D.

In an embodiment, the mycelium may be used as a binder of a food product mixture. In some embodiments, the mycelium may function as an egg replacement. Optionally, as the surface area of the mycelium increases, its' adhesive capacity increases. Optionally, it may be possible to use mycelium alone without the need for additional stabilizers and/or binders.

In an embodiment, the mycelium food product may be stable. In an embodiment, the mycelium food product may be moldable. For example, mycelium mixture may be molded into a particular shape (such as a hamburger patty, chicken nugget, salmon steak, fake shrimp, kebab, sausage, etc.), and may be stable enough to maintain this form throughout a cooking process.

In an embodiment, the food product may have water content of about 40-90%, about 50-80%, about 60-85%, about 65-80%, or about 68-82% w/w, as determined by analysis with a moisture analyzer. Preferably, the food product may have a water content of about 68-82% w/w as determined by analysis with a moisture analyzer. Optionally, this may be an indication of the “juiciness” of the food product.

In some embodiments, a hybrid food product includes a mixture of a processed mycelium as disclosed herein (for example, milled mycelium) and/or processed fruiting bodies, together with processed meat obtained from animal source (i.e., from livestock), or cultured meat (i.e., cultured animal cells or tissues). Optionally, one or more further additives may be added to the hybrid products, including, for example, but not limited to: flavoring, coloring, taste and/or nutrient supplements, protein of other sources, fat of other sources, and the like, or any combination thereof.

According to some embodiments the cultured meat my include cultured animal cells. In some embodiments, the cultured meat may include one or more types of cultured animal cells. In some embodiments, the cultured cells may be of similar or different origin (i.e., from the same animal species, such as, for example, bovine, poultry, fish). In some embodiments, the cultured cells may be of various lineages, such as, for example, but not limited to: myoblasts, fibroblasts, lipoblasts, hepatocytes, osteoblasts, odontoblasts, neuronal progenitor cells, neural stem cells, multipotent stem cells, hematopoietic stem cells, adipo-fibroblasts, adipose-derived stem cells, mesenchymal stem cells, muscle side population cells, circulating skeleton stem cells, neural progenitor cells, multipotent adult progenitor cells, mesodermal progenitor cells, and the like, or any combinations thereof.

According to some exemplary embodiments, a hybrid food product may include various proportions of milled mycelium, chopped fruiting bodies, and ground meat that may be mixed together, optionally, along with one or more various flavoring, coloring, taste and/or nutrient supplements, to produce a hybrid food product. In some embodiments, a hybrid food product may include various proportions of milled mycelium, chopped fruiting bodies, and cultured meat (i.e., cultured cells or tissues) that may be mixed together, optionally, along with one or more various flavoring, coloring, taste and/or nutrient supplements, to produce a hybrid food product.

In an embodiment, the food product may comprise at least about 19 g protein per 100 g hybrid beef burger patty. Optionally, the amount of protein in the food product comprising mycelium have a reduced protein profile in relation to a 100% beef product, but may have a good protein profile when compared to 100% plant-based meat substitute products.

In an embodiment, the food product may comprise at least about 3% dietary fiber. Optionally, between about 2-6%, between about 2.5-5%, preferably about 3-4% dietary fiber

In an embodiment, the food product may comprise about 12% total fat. Optionally, between about 7.5-20%, between about 10-15%, preferably between 11-13% total fat.

In an embodiment, the food product may comprise about 4.5% g saturated fat. Optionally, between about 2.5-10%, between about 3-6%, preferably between 4-5% total fat.

In some embodiments, there is provided a kit for preparing a food product, the kit includes a ready for use mycelium biomass (product), and instructions for use for forming a food product (for example by mixing with one or more additional food ingredients). In some embodiments, the ready for use mycelium biomass may include processed or non-processed mycelium biomass. In some embodiments, the ready for use mycelium biomass may include one or more additional ingredients (such as, for example, but not limited to: fruit bodies, flavorants, colorants, oil, liquid, and the like, or any combination thereof). In some embodiments, the ready for use mycelium may be in any suitable form or shape, such as, for example, but not limited to: powder, grinded, milled, in chunks, etc.

Reference is now made to the figures.

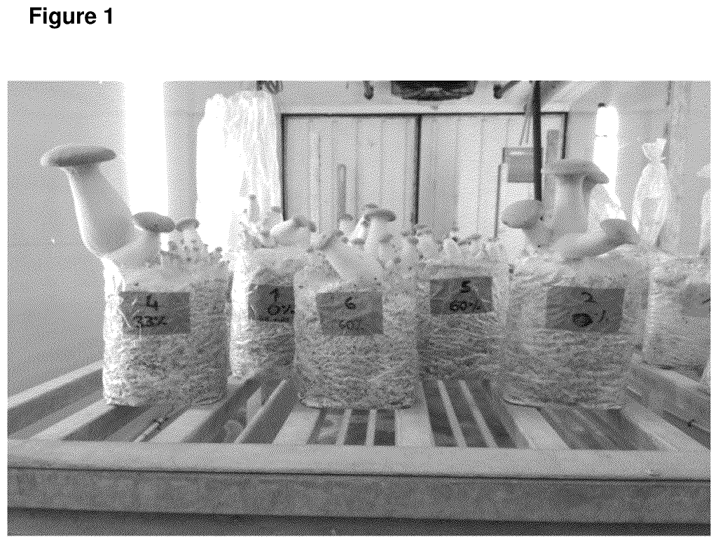

FIG. 1 provides an exemplary image of fungal fruiting bodies with mycelium within a substrate, in accordance with an embodiment of the invention. For example, a standard set up for cultivating fruiting bodies is shown. The fruiting bodies may be harvested according to their maturity levels based on cap opening. However, the mycelium is inculcated with the substrate in which it is grown and is therefore difficult to harvest.

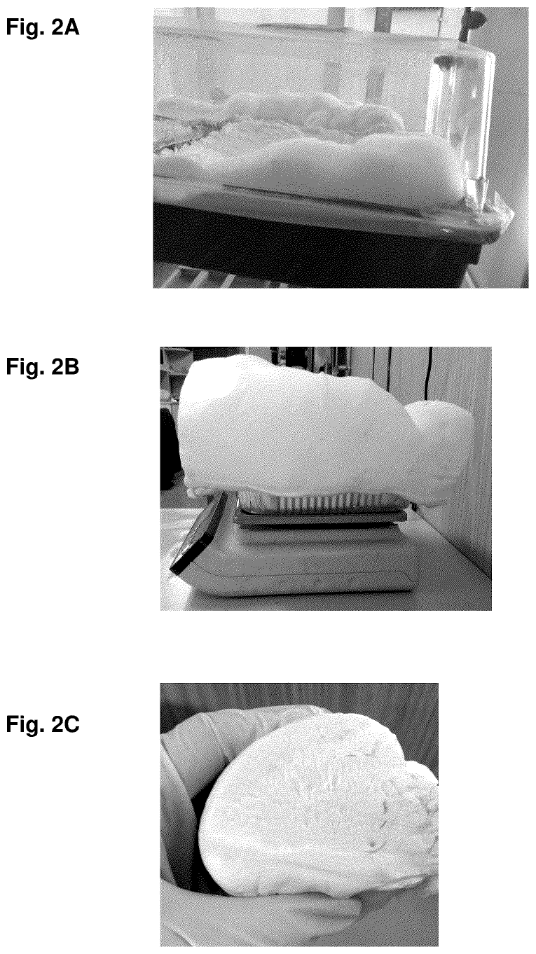

FIG. 2A is an exemplary image of mycelium sheets grown on substrate in a growth chamber, in accordance with an embodiment of the invention. For example, growth trays may be placed in a growth chamber in which the temperature, relative humidity and % CO₂ are controlled. Optionally, the growth chamber may be maintained in the dark for the duration of mycelial growth.

FIG. 2B is an exemplary image of harvested mycelium, in accordance with an embodiment of the invention. For example, the method provided according to some of the embodiments described herein allows for the harvest of a net weight of mycelium without having to separate it from the substrate. Optionally, additional cycles of growth are encouraged by addition of nutrients to the substrate after harvest.

FIG. 2C is an exemplary image of cross-section of harvested mycelium, in accordance with an embodiment of the invention. For example, the harvested mycelium contains little to no substrate material. Optionally, the mycelium may be easily handled, e.g., chopped, ground, milled, freeze-dried, etc. without the need for a tedious separation process from substrate material.

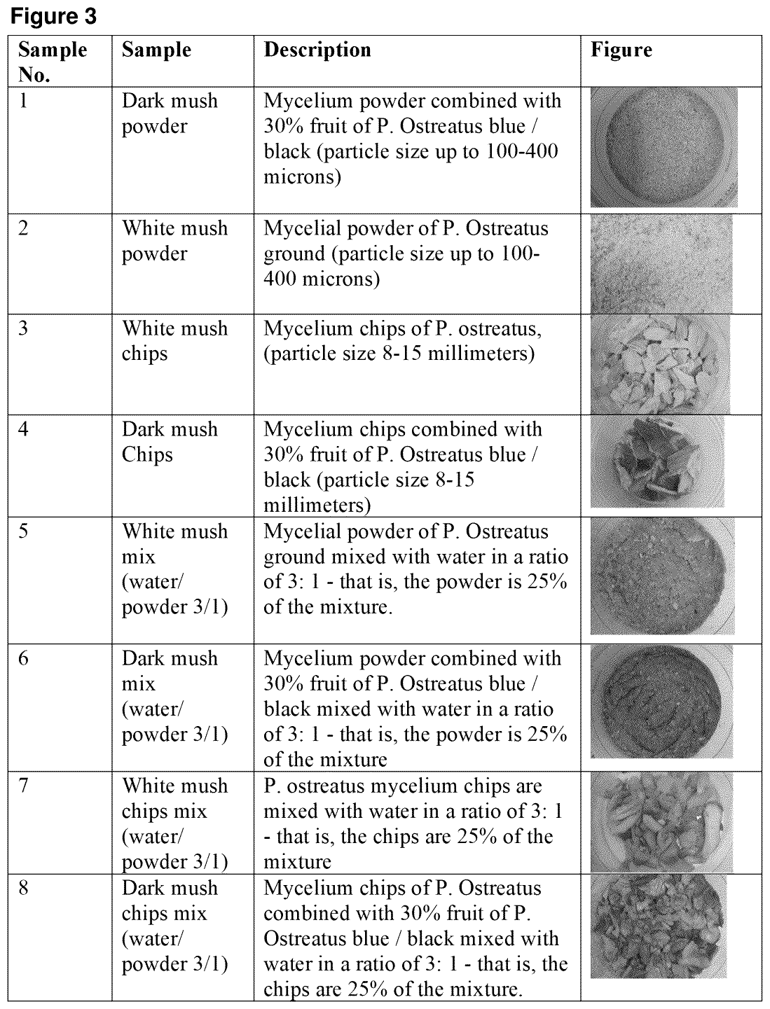

FIG. 3 shows a Table of examples of mycelium (“mush”) powder or mycelium (“mush”) chips, with and without additional fruit bodies and exemplary images thereof, in accordance with an embodiment of the invention. Such mycelium powder or chips may be used for/during the preparation of various food products. Example 9, Table 9 corresponds to the table provided in FIG. 3.

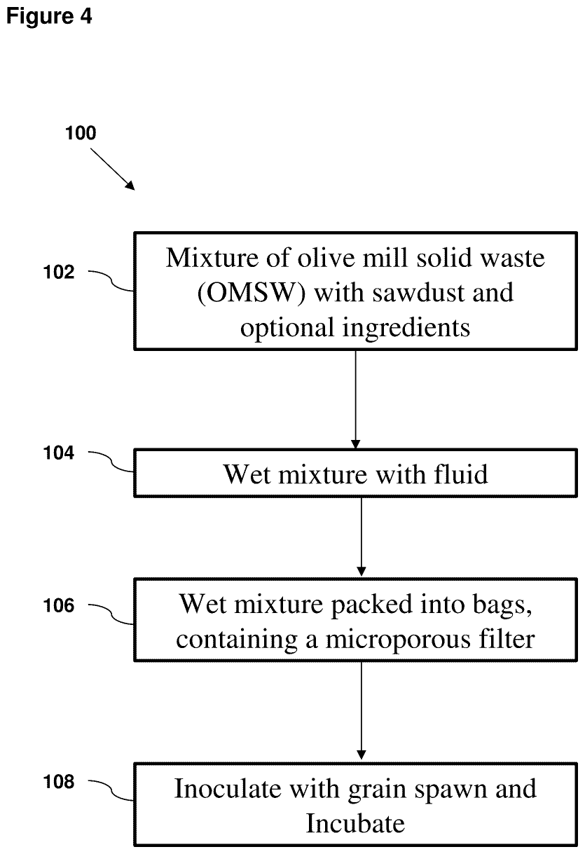

FIG. 4 is a flow diagram of growth of fungal matter in accordance with an embodiment of the invention. In the example shown in FIG. 4, method 100 for growth of fungal matter includes at step 102, preparing a substrate (for example, including a mixture of olive mill solid waste (OMSW) and optional ingredients, such as, sawdust (such as eucalyptus sawdust/ground branches), which is wet with fluid (for example, water). Next, at step 104, the wet mixture is packed into bags (for example, polypropylene bags), containing a microporous filter. and the bags may be sterilized (for example, in an autoclave). The sterilized bags are then inoculated with grain spawn and allowed to incubate, at step 106.

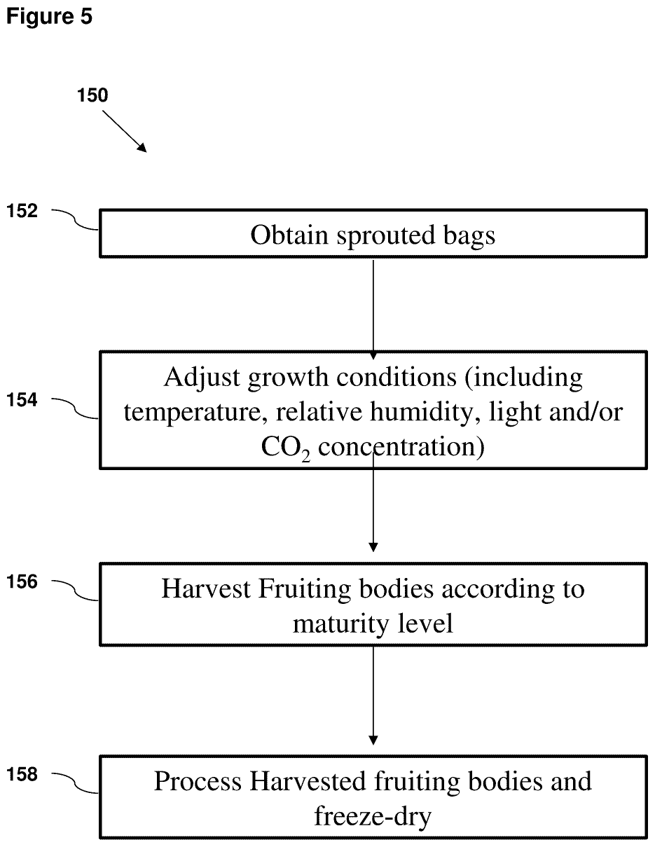

FIG. 5 is a flow diagram of method for growing of fruiting bodies, in accordance with some embodiments. In the example shown in FIG. 5, method 150 includes obtaining at step 152 sprouted bags (for example, obtained as described with respect to FIG. 4, according to method 100). The bags are opened and the growth conditions, such as temperature, relative humidity, light and CO₂ concentration are adjusted/controlled (step 154). The resulting fruiting bodies are harvested in step 156 according to maturity level (e.g., size, cap opening, etc.). The harvested fruiting bodies are chopped into small pieces and freeze-dried for later use (step 158).

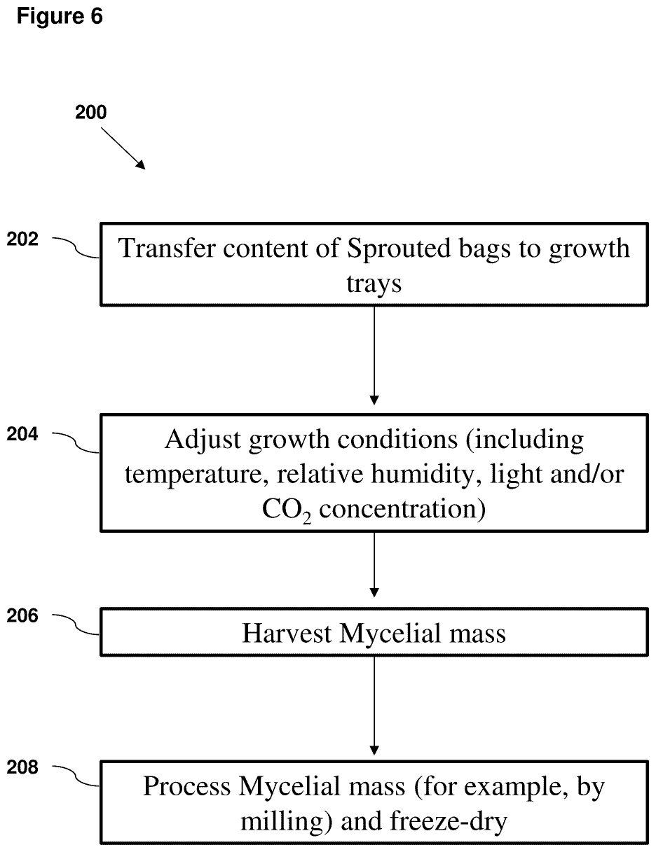

FIG. 6 is a flow diagram of method for growth of mycelial matter in accordance with an embodiment of the invention. In the example shown in FIG. 6, method 200 includes obtaining at step 202 the sprouted bags (for example, obtained as described with respect to FIG. 4, according to method 100). The bags are opened and contents may be transferred to growth trays. The growth trays may be placed in growth chambers where the growth conditions such as temperature, relative humidity, light (absence of light) and CO₂ concentration are controlled (step 204). The resulting mycelial mass may then be harvested at step 206, and further processed (for example, by milling) and freeze-dried for later use (step 208).

Unless otherwise defined, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein, the denotation “% w/w” refers to weight percent of the subject of the statement relative to the total weight of the dairy composition.

As used herein, the denotation “% dw/dw” refers to dry weight percent of the subject of the statement relative to the total dry weight of the dairy composition.

As used herein, the term “meat” encompass meat obtained from animals (for example, pork, fish, poultry, bovine, cattle, etc.) and/or cultured meat obtained from cultured animal cells and/or cultured animal tissues. In some embodiments, the term “cultured meat” is interchangeable with “synthetic meat” and “cell-cultured meat” and is directed to meat cells or tissue grown in-vitro in cell culture (in culture plates, tissue scaffolds, etc.).

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The terms “comprises”, “comprising”, “includes”, “including”, “having” and their conjugates mean “including but not limited to”.

The term “consisting of” means “including and limited to”.

The term “consisting essentially of” is defined such as that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### EXAMPLES

### Example 1: Mycelium Growth Conditions

The Pleurotus sp. mycelium was grown on a sterilized mixture of eucalyptus sawdust (ground branches), beer cereal waste and olive mill solid waste (OMSW). The OMSW is the solid fraction from a three-phase olive mill. OMSW was added at concentrations from 33% (dw/dw) up to 80% (dw/dw). The mixture was wetted to 58% water content and packed into 15 L polypropylene bags, containing a microporous filter (Unicorn 14A), 2 kg wet substrate/bag. The bags were autoclaved at 121° C. for 1 h, and cooled to 25° C. for inoculation with grain spawn.

For mycelium development, content of sprouted bags was transferred to growth trays, inserted into growth bags (Unicorn 10A) and incubated at 25° C. for 10-14 days. When the trays were fully inoculated with the mushroom mycelium, they were taken out of the bags, and put in growth chambers. The conditions were temperature of 25° C., relative humidity 100%, in dark, approximately 5% CO₂ concentration and irrigation with Sprinklers (Netafim CoolNet 7.5 l/h)

Yield of P. ostreatus mycelium (strain: Sylvan k-12), grown on sterilized substrate mixture of eucalyptus sawdust (33%) beer cereals (33%) and olive mill solid waste (33%) in trays, 500 g substrate/tray, 4 replicates for treatment. The results are presented in Table 1 below:

TABLE 1

| | Yield |
|---|---|
| Growth chamber | (w/w % ± sd) |
| Propagator 55 liter | 27.0 ± 4.4 |
| Propagator 55 liter | 30.2 ± 2.5 |
| Growing chamber 78 liter | 55.5 ± 26.3 |
| Growing chamber 78 liter | 34.4 ± 15.6 |

### Example 2: Mycelium Growth of Two Species of Pleurotus on Different Substrates Mixture

The mushrooms mycelium were grown on a sterilized mixture of organic waste. The mixture was wetted to 50-70% water content and packed into autoclavable containers, containing a microporous filter (Sac O2 O118/80+OD118), sterilized in an autoclave at 121° C. for 1 h, cool to 25° C. and inoculate with grain spawn. The containers incubate at 23.5° C. for 14-21 days and once fully inoculated with the mushroom mycelium, the lids removed, and the containers were put in growth bags (Unicorn 4A) at temperature of 25° C., in dark.

Yield of P. ostreatus (strain: Sylvan k-12) and P. colombinus (strain Sylvan 3030) mycelium, grown on different substrates mixture in 565 ml containers, 250 g substrate/container, 3 replicates for treatment.

The results are presented in Table 2 below:

TABLE 2

| | | Mycelium yield |
|---|---|---|
| Mushroom | | (w/w substrate) |
| strain | Substrate composition | (%) |
| P. ostreatus | 33% eucalyptus + 33% beer cereals + 33% OMSW | 1.0 |
| (strain: Fungisem | 33% eucalyptus + 33% beer cereals + 33% Strauss coffee | 0.3 |
| K-12) | 33% eucalyptus + 33% beer cereals + 33% Nespreso | 0.8 |
| | coffee | |
| | 33% eucalyptus + 33% wheat bran + 33% OMSW | 2.7 |
| | 33% eucalyptus + 33% beer cereals + 33% soy pulp | 1.3 |
| P. colombinus | 33% eucalyptus + 33% beer cereals + 33% OMSW | 3.7 |
| (strain: Sylvan | 33% eucalyptus + 33% beer cereals + 33% Strauss coffee | 1.8 |
| 3030) | 33% eucalyptus + 33% beer cereals + 33% Nespreso | 7.1 |
| | coffee | |
| | 33% eucalyptus + 33% wheat bran + 33% OMSW | 2.0 |
| | 33% eucalyptus + 33% beer cereals + 33% soy pulp | 2.9 |

Further substrate compositions used and humidity levels thereof, are presented in Tables 3-4 below:

TABLE 3

| | 1 | 2 | 3 | 4 | 5 | 6 |
|---|---|---|---|---|---|---|
| Ingredients (%) | | | | | | |
| ground eucalyptus | 33.33 | 15 | 15 | 15 | 20 | 25 |
| branches | | | | | | |
| beer cereals | 33.33 | 40 | 45 | 42.5 | 40 | 37.5 |
| olive mill solid waste | 33.33 | 45 | 40 | 42.5 | 40 | 37.5 |
| Ingredients (gr) | | | | | | |
| ground eucalyptus | 194.6 | 94.4 | 90.0 | 87.8 | 123.0 | 151.3 |
| branches | | | | | | |
| beer cereals | 194.6 | 251.6 | 270.0 | 248.6 | 246.0 | 226.9 |
| olive mill solid waste | 194.6 | 283.1 | 240.0 | 248.6 | 246.0 | 226.9 |
| water (ml) | 716.0 | 671.0 | 700.0 | 715.0 | 685.0 | 695.0 |
| # substrate moisture (%) | 55.1 | 51.6 | 53.8 | 55.0 | 52.7 | 53.5 |
| dry mater | 584.0 | 629.0 | 600.0 | 585.0 | 615.0 | 605.0 |
| measured humidity (%) | 60.7 | 61.3 | 60.2 | 57.2 | 54.5 | 56.5 |

TABLE 4

| | 1 | 2 | 3 | 4 | 5 |
|---|---|---|---|---|---|
| Ingredients (%) | | | | | |
| ground eucalyptus | 33.33 | 25 | 30 | 35 | 40 |
| branches | | | | | |
| beer cereals | 33.33 | 50 | 40 | 30 | 20 |
| olive mill solid waste | 33 | 25 | 30 | 35 | 40 |
| Ingredients (gr) | | | | | |
| ground eucalyptus | 195 | 140 | 172 | 207 | 242 |
| branches | | | | | |
| beer cereals | 195.0 | 279.5 | 229.8 | 177.1 | 121.2 |
| olive mill solid waste | 195 | 140 | 172 | 207 | 242 |
| water (ml) | 714.9 | 741 | 725.4 | 709.8 | 694.2 |
| # substrate moisture | 54.99 | 57.00 | 55.80 | 54.60 | 53.40 |
| (%) | | | | | |
| dry mater | 585.0715 | 559 | 574.6 | 590.2 | 605.8 |

### Example 3: Fruit Bodies Yield of Some Pleurotus Species

The mushrooms were grown on a sterilized mixture of eucalyptus sawdust (60%) and olive mill solid waste (40%). The mixture was wetted to 54% water content and packed into autoclavable bags, containing a microporous filter (Unicorn 14A), 3 kg wet substrate/bag. The bags were autoclaved at 121° C. for 1 h, and cooled to 25° C. for inoculation with grain spawn. The bags incubate at 23.5° C. for 14-21 days. For fruiting, the bags were opened, and the temperature was reduced to 18° C. with a relative humidity of 90%, 8 h daily light.

Fruit bodies are freshly harvest according to their maturity level (cap opening).

yield (%) determine by: wet fruit bodies weight/wet substrate weight ×100

Yields (fruit bodies) of some Pleurotus species, grown on sterilized substrate mixture of organic waste (5 replicates for treatment) (w/w %). The results are presented in Table 5 below:

TABLE 5

| Species | Commercial strain | Yield |
|---|---|---|
| P. ostreatus | Sylvan k-12 | 16.43 |
| P. colombinus | Sylvan 3030 | 13.23 |
| P. pulmonarius | Sylvan 3014 | 18.63 |
| P. sajor caju | Jamaica 503 | 18.44 |
| P. cornucopiae | Sylvan 3040 | 18.04 |
| P. salmoneo | Mycelia 2780 | 31.45 |

### Example 4: Mushroom Growth and Harvesting

The mushrooms were grown on a sterilized mixture of organic waste. The mixture is wetted to 60-70% water content and packed into autoclavable containers, containing a microporous filter (Sac O2 O118/80+OD118).

The containers are sterilized in an autoclave at 121° C. for 1 h, cool to 25° C. and inoculate with grain spawn. The containers incubate at 23.5° C. for 14-21 days. For fruiting, the containers were opened, and the temperature was reduced to 18° C. with a relative humidity of 90%, 8 h daily light.

Fruit bodies are freshly harvest according to their maturity level (cap opening). Yield (%) was determined by: wet fruit bodies weight/wet substrate weight ×100.

The results are presented in Table 6 below:

TABLE 6

| | Fruit bodies |
|---|---|
| | yield* (w/w |
| | substrate) |
| Substrate composition | (%) |
| 33% eucalyptus + 33% beer cereals + 33% OMSW | 31.3 |
| 33% moringa + 33% beer cereals + 33% Nespreso coffee | 34.9 |
| 33% eucalyptus + 33% beer cereals + 10% Nespreso | 28.7 |
| coffee + 23% OMSW | |
| 33% eucalyptus + 33% beer cereals + 23% Nespreso | 31.7 |
| coffee + 10% OMSW | |
| 33% eucalyptus + 33% wheat bran + 33% Nespreso coffee | 29.4 |
| 33% eucalyptus + 33% beer cereals + 33% Strauss coffee | 25.3 |
| 33% eucalyptus + 33% beer cereals + 33% Strauss coffee | 26.8 |
| 33% eucalyptus + 33% beer cereals + 15% Strauss | 32.3 |
| coffee + 15% OMSW | |

*Yields of Pleurotus ostreatus fruit bodies, grown on different substrates mixture in 565 ml containers, 250 g substrate/container, 4 replicates for treatment.

### Example 5: Glucans Analysis

The glucan content of dried Pleurotus sp. fruiting bodies was determined using a mushroom and yeast specific β-glucan kit (Megazyme International, Wicklow, Ireland) based on a colorimetric reaction. The analysis was conducted according to the manufacturer's instructions. The enzyme kit contains exo-1,3-β-glucanase, β-glucosidase, amyloglucosidase and invertase; glucose determination reagent (glucose oxidase peroxidase, and 4-aminoantipyrine), and glucose standard solution. Total-glucan, α-glucan, and β-glucan were measured in the samples. Measurement of total glucan content is performed following a previous solubilization in ice cold 12 M H₂SO₄ of the Pleurotus sp. dried samples and then hydrolysis to near completion in 2 M H₂SO₄ for 2 h at 100° C. Subsequent to neutralization with 2 M potassium hydroxide, glucose hydrolysis is accomplished using a mixture of exo-1,3-β-glucanase and β-glucosidase in sodium acetate buffer (pH 5.0) for 1 h at 40° C. The absorbance of the resulting color was measured at 510 nm using a spectrophotometer (Synergy 2, Multi-Mode Reader, BioTek, Winooski, VT, USA). Total Glucan (% dw/dw) and α-Glucan (% dw/dw) were calculated, and by difference between those two, the β-glucan content (% dw/dw) was finally calculated. Glucan content was expressed as percentage (dw/dw) of the fruiting body dry weight. The control provided by the kit consists of glucans extracted from Saccharomyces cerevisiae.

RNA Extraction from the Fruit Body Tissue, from the Fungus Pleurotus eryngii

The fruiting bodies were frozen at −20° C. immediately after harvesting, then freeze-dried and stored until RNA extraction according to the commercial kit protocol (SV Total RNA Isolation System Promega). Approximately 30 mg were taken from the fruit body tissue, which was crushed with liquid nitrogen, the RNA quality was tested and the samples were frozen at −80° C.

cDNA Synthesis

Complementary DNA synthesis was performed using a kit and the High-Capacity cDNA Reverse Transcription Kits protocol from “Thermo Fisher”.

### Q-PCR

Q-PCR reaction of the composition of cDNA 4 μL, Fast SYBR Green Master mix 10 μL, DDW 4 μL and a mixture containing specific primers (table 7). Final reaction volume of 20 μL was placed in a Bio-Rad CFX instrument with Maestro CFX software and standard method. Comparative Ct for Real-Time PCR analyzes were performed in three repetitions for each gene, a NTC (Non-Template Control) test was performed in parallel for each gene. GAPDH served as the normalizing gene.

### Primer for Q-PCR Analysis

TABLE 7
Primers list for Q-PCR analysis on the poly-
saccharides goal genes α and β Glucan from
the fungi Pleurotus eryngii

| Gene | Primer Reverse | Primer Forward |
|---|---|---|
| AGS6 | GCCGTAGGGTCGAAATGGTCA | TTGCTACACCCTCGGATCC |
| | AG (SEQ ID NO: 1) | GAA (SEQ ID NO: 2) |
| BGS1 | AAGAGTGCAGGCAAAACGAT | CAACAATGCTTTTCTGGCT |
| | (SEQ ID NO: 3) | TC (SEQ ID NO: 4) |
| GAPDH | GTTAACACTACGACCTCCACG | ACCTCGAGACTTACGACCC |
| | (SEQ ID NO: 5) | G (SEQ ID NO: 6) |

### Example 6: Growth Cycle—Pleurotus eryngii (About 5 Weeks)

The growing media containing OMSW, beer cereals (BC), eucalyptus sawdust (Euc) paste and water in various ratios (Table 1), were placed in growing bags with a gas passage filter and sterilized in an autoclave (100-121° C.) for two hours. The bags were cooled to room temperature, inoculation under sterile conditions with seed P. eryngii Zen-3069 from “Sylvan” (3% of the substrate weight) and mixed homogeneously. The bags were placed for germination in a controlled room at a temperature of 23-34° C. and a humidity of about 50-60%. At the end of the germination phase, the tops of the bags were opened and they were transferred to a growing room for fruiting bodies, at a temperature of 18° C. and 80-90% humidity. The fruiting bodies from each treatment were dried separately with a lyophilizer, mixed and placed in a freezer at −20° C.

TABLE 8
The four experimental groups of growth
media in varying concentrations of OMSW

| SUBSTRATE | 1 | 2 | 3 | 4 |
|---|---|---|---|---|
| OMSW % | 0 | 33 | 60 | 80 |
| Beer cereals (%) | 50 | 33 | 20 | 10 |
| Eucalyptus sawdust (%) | 50 | 33 | 20 | 10 |
| Moisture (%) | 60 | 58 | 54 | 52 |

### Example 7: Test for Nitrogen Levels Using the Kjeldahl Method

After the growth of the fungal cycle was completed, the fruit bodies were dried in an oven at 60° C. and ground to a size of 1 mm. Then, 0.5 g were weighed from each of the test groups (in duplicates), and transferred to test tubes.

150 mg of Kjeldahl Catalyst (selenium reagent mixture) was added to each test tube, in duplicate per sample. After transferring the test tubes to a chemical fume hood, 10 ml of 98% sulfuric acid and 10 ml of 32% hydrogen peroxide were added. The digestion process lasted one and a half hours, at a temperature of 400° C. in a Buchi K-435 digestor and thereafter cooled for 30 minutes at room temperature. The boron acid H₃BO₃ 2% was titrated in 0.1N HCl in the Metrohm 719S titrator.

### Example 8: Examining General Protein Concentration Levels Using Bradford

After the end of the growth of the fungal cycle, the fruiting bodies were frozen at −20° C., then freeze-dried and stored at −20° C. 20 mg of the dry powder was then weighed and crushed in a MIXER MILL 400 MM device for one minute for each experimental group (control and treatments). Then 400 μl of distilled water was added to this and it was further ground for 2 minutes. The test samples were then centrifuged for 15 minutes at 14,000 rpm at 4° C. for 2 minutes. From each test tube a suspension of volume 5 μL was taken and diluted with distilled water to a volume of 85 μL (18×). From the same suspension dilutions of the fungal tissues were taken for each 5 μL and mixed together with 200 μL of Bradford 5× reagent) until a permanent color was obtained. To create the calibration curve, 7 series were mixed (at concentrations of BSA 2, 1.5, 1, 0.75, 0.5, 0.25, 0.125 mg/ml), from each concentration each 5 μL was taken and mixed together with 200 μL of Bradford reagent (5×) and measured on an “INFINITE 200 PRO” OD device at a wavelength of 595 nm.

### Example 9: Color Test Summary for Mycelium

Following a number of trials and uses of the raw material, it was discovered that incorporating certain fruiting bodies into mycelium can change the color of the powder, thus better adapting to specific applications. For example: the mycelium itself is white. Mixing it with water causes the color to darken, but it still remains a shade of light yellow. On the other hand, if blue/black Pleurotus ostreatus fruit bodies are added to the mycelium, a darker color is obtained even as a dry powder. The color darkens even more when mixed with water to a brown color that is more suitable for the color of cooked/roasted beef.

Optionally, a pink mycelium, mixed with Pleurotus salmoneo may be obtained, which may be added to salmon and/or beef.

This can also be seen with the naked eye and using a spectrophotometric method on a ColorFlex EZ from HunterLab.

The samples are present in FIG. 3

The following spectrophotometric results (Table 3) are based on the Hunter L,a,b color space which is a 3-dimensional rectangular color space based on Opponent-Colors Theory. L (lightness) axis—0 is black, 100 is white, and 50 is middle gray a (red-green) axis—positive values are red, negative values are green, and 0 is neutral b (blue-yellow) axis—positive values are yellow, negative values are blue, and 0 is neutral.

TABLE 9
Hunter L.a.b. Color Test Results

| No. | ID | L* | a* | b* | a*/b* |
|---|---|---|---|---|---|
| 1 | Dark mush powder | 75.31 | 1.25 | 18.79 | 0.07 |
| 2 | White mush powder | 81.56 | 0.6 | 22.73 | 0.03 |
| 3 | White mush chips | 76.14 | 1.24 | 18.15 | 0.07 |
| 4 | Dark mush Chips | 67.12 | 2.42 | 19.98 | 0.12 |
| 5 | White mush mix (water/powder 3/1) | 56.78 | 5.35 | 36.18 | 0.15 |
| 6 | Dark mush mix (water/powder 3/1) | 45.33 | 4.69 | 26.8 | 0.18 |
| 7 | White mush chips mix (water/powder | 57.26 | 2.31 | 25.69 | 0.09 |
| | 3/1) | | | | |
| 8 | Dark mush chips mix (water/powder | 49.75 | 4.28 | 24.92 | 0.17 |
| | 3/1) | | | | |

### Conclusions:

The sample including only mycelium exhibits a brightness of 81, while the sample of mycelium with the addition of dark fruit bodies exhibit a brightness of 75. The same applies after adding water: the mycelium pattern is at a brightness of almost 57 while the mixture of mycelium+dark fruit+water is at a brightness of 45.

### Example 10: Food Texture and Flavor

The aim of the experiment was to obtain as similar as possible texture as that of meat (beef or chicken). In addition, the experiment aims to test the ability of mycelium to function as a binder on its own without the need for other supplements.

In addition, the experiment aims to determine a ratio of meat to mycelium in a various food product such as, hamburger and chicken nuggets.

### Experimental Results:

Several methods were used: soaking whole dried mushrooms in water at a weight ratio of 75:25, respectively, mixing ground mycelium with water at a ratio of 75:25 and roasting it in a pan, and mixing mycelium powder with water at a ratio of 70:30 respectively.

In general, the finer ground the mycelium, the higher the adhesion capacity, the more uniform the texture of the meat patty becomes, and it is not possible to distinguish between mycelium and meat.

For chicken nuggets and chicken patties no egg was used and the mycelium (after mixing mycelium powder with water in a ratio of 70:30, respectively), bound the ground chicken well without other additives. Without wishing to be bound to any theory or mechanism, this may be due to the high content of globulin and albumin proteins present in the mycelium.

In a product based on whole mushrooms a ratio of 33% mixture of mush (mycelium+water) 67% meat was unsuccessful: the patty fell apart and the taste of the mushrooms was noticeable.

In contrast, in the chicken patties product, where mycelium powder was used, even when the ratio was 50% mush mixture and 50% meat—the product was normal, uniform in texture, delicious, and without aftertaste.

As the surface area of the mycelium increases, the adhesive capacity increases and it is possible to use mycelium alone without the need for additional stabilizers/binders.

### Example 11: Comparing Nutritional Values to a Hybrid Hamburger Versus a 100% Beef Burger

The aim of the experiment was to determine nutritional values of a hybrid hamburger versus a 100% beef burger. The results indicate that in general, the addition of mycelium results in reduced fat and saturated fat, and the addition of dietary fiber.

Regarding protein: addition of mycelium results in reduced protein in relation to a 100% beef product, but may have a good protein profile when compared to 100% plant-based products.

The results are presented in Table 10:

TABLE 10
Comparative nutritional analysis

| | Cooked | Cooked hamburger | Cooked hamburger |
|---|---|---|---|
| | hamburger | patty 75% beef, | patty 67% beef, |
| | patty - 100% | 25% mush mixture | 33% mush mixture |
| | meat | (mycelium + water) | (mycelium + water) |
| Total Fats | 15.0 | 11.5 | 10.4 |
| Saturated fats | 6.0 | 4.5 | 4.1 |
| Trans fats | <0.5 | <0.5 | <0.5 |
| Sodium | 1000.0 | 1000.0 | 1000.0 |
| Carbohydrates | 0.1 | 4.1 | 5.3 |
| Dietary fiber | 0.0 | 1.5 | 2.0 |
| Sugar | 0.1 | 0.1 | 0.1 |
| Total Protein | 20.0 | 17.0 | 16.1 |

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

1.-28. (canceled)

29. A mycelium derived from a cultivation substrate comprising olive mill solid waste (OMSW) at a concentration of about 30-60%, said mycelium comprises at least about 35% w/w of total Glucan and at least about 6.5% w/w of total protein.

30. The mycelium of claim 29, wherein the mycelium is aerial mycelium.

31. The mycelium according to claim 29, further comprising at least about 40 μg/g of Vitamin D and/or at least about 2.8% w/w Nitrogen.

32. The mycelium according to claim 29, wherein the Glucans comprises at least about 19% beta-Glucan and at least about 5% alpha-Glucan.

33. The mycelium according to claim 29, wherein the yield of the mycelium is at least about 25% w/w.

34. The mycelium according to claim 29, wherein the mycelium is from the species Pleurotus osteratus, Pleurotus eryngii, Pleurotus sp. Pleurotus.colombinus, Pleurotus pulmonarius, Pleurotus sajor caju, Pleurotus cornucopiae, or Pleurotus salmoneo.

35. The mycelium according to claim 29, for use as a binder of a food product mixture.

36. The mycelium according to claim 29, in a powdered form.

37. The mycelium according to claim 36, wherein the mycelium is grinded to a particle size of about 0.2 mm to 200 mm.

38. A composition comprising a mixture of at least one aerial mycelium.

39. The composition according to claim 38, comprising at least two aerial mycelium, optionally each derived from a different species.

40. The composition according to claim 39, wherein at least one of the species is Pleurotus osteratus, Pleurotus eryngii or Pleurotus sp.

41. A food product comprising a mixture of:
a) a mycelium according to claim 29; and
b) at least partially ground meat.

42. The food product according to claim 41, wherein the mycelium is grinded prior to mixing with the meat.

43. The food product according to claim 41, wherein the aerial mycelium is mixed with water at a ratio of about 20:80, about 25:75, about 40:60, about 50:50 w/w prior to mixing with the meat.

44. The food product according to claim 41, wherein the weight ratio between the mycelium and the meat is about 10:90, about 20:80, about 25:75, about 40:60, about 50:50, about 60:40, about 75:25 about 80:20, or about 90:10 w/w.

45. The food product according to claim 41, further comprising one or more of: oil, flavor enhancer and colorant.

46. The food product according to claim 41, wherein the food product comprises one or more of: at least about 2% dietary fibers (w/w), less than about 15% fat (w/w) and/or less than about 5% saturated fat (w/w).

47. The food product according to claim 41, wherein the food product is stable and/or moldable.

48. The food product according to claim 41, wherein the food product is essentially homogenous and/or having a water content of about 65-85% w/w.
